(19) United States
(12) Patent Application Publication
(10) Pub. No.: US 2014/0276082 A1
(43) Pub. Date: Sep. 18, 2014

(54) ULTRASOUND TRANSDUCER WITH NEEDLE CHANNEL

(71) Applicant: Lumoptik LLC, Shaker Heights, OH (US)

(72) Inventors: Arthur A. Janicki, Cincinnati, OH (US); Oscar Thomas Janicki, North Olmsted, OH (US)

(73) Assignee: LUMOPTIK LLC, Shaker Heights, OH (US)

(21) Appl. No.: 14/208,233

(22) Filed: Mar. 13, 2014

Related U.S. Application Data
(60) Provisional application No. 61/782,941, filed on Mar. 14, 2013.

(51) Int. Cl. A61B 8/08; A61B 8/00

(52) U.S. Cl. CPC A61B 8/0833; A61B 8/4444
USPC 600/461

(57) ABSTRACT

An ultrasound probe system for guiding introduction of an instrument into a patient includes a disk having a surface and an open channel passing axially through the disk and at least two ultrasound transducer elements disposed on the surface of the disk in a parallel arrangement. The open channel is configured to receive the instrument therethrough to permit alignment of the instrument with respect to the disk. Each of the ultrasound transducer elements is configured to transmit and receive ultrasonic waves for detecting an anatomical structure. A graphical representation of at least a portion of the anatomical structure can be provided for guiding the introduction or insertion of the instrument into the anatomical structure.

This application claims priority under 35 U.S.C. § 119 to U.S. Provisional Application No. 61/782,941, which was filed on Mar. 14, 2013, and is herein incorporated by reference in its entirety.

### BACKGROUND

Percutaneous introduction of the needles and catheters into the deep vessels (jugular, subclavian, femoral and other) requires detailed knowledge of the anatomy of the region and specialized training.

The insertion can be associated with numerous potential complications including: bleeding, lacerations of the neighboring arteries or veins, injury to the nerves, pneumothorax and death.

Recently, use of ultrasound for guiding the insertion has improved the safety of those procedures. However, presently available ultrasonic transducers and ultrasonic systems require triangulation of the needle insertion in relation to the ultrasonic image. Further, the quality of the image presented to the user during such procedure is poor (grainy and with poor resolution).

Thus, additional ultrasonic image interpretation training is necessary for any user attempting to perform ultrasound guided insertion of the needle.

### SUMMARY

Various embodiments are directed to a disk-shaped probe having an ultrasonic transducer and a central channel or opening within the disk to accommodate a needle (including, for example, a penetration sensor-equipped needle) or other instrument, such as a catheter. The probe may be used for guided introduction or insertion of the instrument, via the central channel, into a vessel or other anatomical structure of a patient. Some embodiments provide a computer-enhanced graphic image of the vessels and other structures in the area covered by ultrasonic probe. The image may be used, for example, by a user for manually positioning and orienting the instrument, using the probe, with respect to the target structure so that the tip of the instrument can be introduced or inserted into the desired area.

The size and location of the structures in the image can change as the user moves the probe around the area to determine the optimum needle insertion point and/or angle. Additionally, in some embodiments, a crosshair, or other suitable symbol, can be located in the center of the image indicating exact point of the penetration of the vessel or other structure when the needle is inserted through the channel in the center of the transducer. In some embodiments, data from the sensor-equipped needle can be transmitted during insertion of the needle and incorporated into the image on the screen, which gives a user indication that the tip of the needle has reached the lumen of the vessel.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a perspective view of an example of an ultrasonic transducer, in accordance with an embodiment.

FIG. 2A is a perspective view of the ultrasonic transducer of FIG. 1.

FIG. 2B depicts one example of a graphical image representing a structure detected by the ultrasonic transducer of FIG. 1, in accordance with an embodiment.

FIG. 3A is a perspective view of the ultrasonic transducer of FIG. 1.

FIG. 3B depicts another example of a graphical image representing a structure detected by the ultrasonic transducer of FIG. 1, in accordance with an embodiment.

FIG. 4A is a perspective view of the ultrasonic transducer of FIG. 1.

FIG. 4B depicts yet another example of a graphical image representing a structure detected by the ultrasonic transducer of FIG. 1, in accordance with an embodiment.

FIGS. 5, 6 and 7 are different perspective views of an example of an ultrasonic transducer, in accordance with an embodiment.

### DETAILED DESCRIPTION

FIG. 1 depicts an example of a needle-accommodating ultrasonic probe, or transducer 100, according to an embodiment. It will be appreciated that, according to various embodiments, various instruments, such as needles, catheters and the like, can be utilized, and the present disclosure is not intended to limit such instruments to needles. The transducer 100 may include, for example, a flat disk approximately 2 cm to 5 cm thick connected by the lateral power/data cord 102 to an external ultrasound unit 104. The thickness of the disk can be varied to accommodate various needles or other instruments such that the needle or instrument is stably aligned when inserted through the disk for guiding the introduction of the needle or instrument into an anatomical structure of a patient, such as described below.

The disk/transducer 100 can contain two or more linear arrays 106 of crystal (also referred to herein as transducer elements) that emit and then receive ultrasonic sound waves. For example, the ultrasonic transducer 100 may include a disk that contains two or three linear arrays 106 that are parallel to each other and located approximately 1 cm apart. The disk/transducer 100 includes an opening 108 or channel passing through the disk for accommodating the passage of a needle or other instrument, and a sterile channel, sleeve, sheet or other material (not shown in FIG. 1) for separating the transducer disk 100 from the instrument and for providing a sterile environment for the portion of the instrument passing through the opening 108. The opening 108 may, for example, be oriented substantially perpendicular to a surface 112 of the disk/transducer 100. In some embodiments, the disk 100 includes a split 110 forming two portions or halves that can be separated from each other. The split 110 may pass through or adjacent to the opening 108, as shown, for example, in FIG. 5.

FIG. 2A depicts another view of the transducer 100, and FIG. 2B depicts an example of a graphical image 200 generated from signals received by the transducer. The arrays 106 can be configured to fire sequentially such that each array 106 transmits and receives ultrasonic signals independently of and without interference by the other arrays. The ultrasound image can be rendered as two or three images 202, 204, 206 of the area underneath the transducer 100. Each image 202, 204, 206 may, for example, include a graphical representation of at least a portion of the vessel or other structure detected by the corresponding array 106. The graphical representation may be, for example, an artificial or simplified representation of the actual vessel 120 or other structure being imaged (e.g., not a literal representation of the actual vessel).

The images 202, 204, 206 can be summarized (e.g., in an overlay or additive manner) on a display 208 and presented in the form of graphic image of the target area (e.g., the vessel 120 or other structure) by a processor. If the arrays 106 are aligned at approximately 90 degrees to the longitudinal axis 122 of the vessel 120, the images 202, 204, 206 will be almost identical. The images 202, 204, 206 can then be graphically summarized on the screen 208 showing a segment of the vessel 120 user is trying to access and a cross-hair 210 or other marker indicating an executable needle insertion site. The cross-hair 210 may, for example, indicate the point of penetration of the needle or instrument into the vessel or other structure if the needle is inserted through the opening 108. If the transducer 100 is not aligned at 90 degrees to the longitudinal axis of the vessel, the graphic summary will not be displayed and there will be no executable needle insertion site, such as shown in FIGS. 3A and 3B (zero degrees), and FIGS. 4A and 4B (between zero and 90 degrees), where the disk/transducer 100 is oriented at an angle other than 90 degrees. In use, manual manipulation of the transducer 100 for adjusting the position and angle can ultimately align longitudinal axis of the vessel at 90 degrees to the transducer and that will create executable needle insertion site.

In some embodiments, the disk/transducer 100 can be constructed to orient the needle at substantially perpendicular to (e.g., approximately 90 degrees) or at an angle other than 90 degrees (e.g., any angle between zero and 90 degrees) with respect to the longitudinal axis of the vessel 120 or other structure. For instance, the opening 108 may be formed at an angle other than 90 degrees with respect to the surface 112 of the disk/transducer 100.

FIGS. 5, 6 and 7 are perspective views of various examples of the transducer 100. In some embodiments, the disk/transducer 100 has open channel 108 at the center able to accommodate sterile sheet or sterile tube 130 to allow insertion of a needle 140 or catheter in the sterile fashion. The channel 108 is at 90 degrees to the surface plane 112 of the disk/transducer 100. The disk/transducer 100 can, in some embodiments, be divided into hinged halves, or other suitable portions of the circle allowing opening of the disk/transducer 100 to allow insertion of the sterile sheet or tube 130 into the center opening 108 of the disk/transducer 100, after such insertion the disk/transducer 100 can be closed and ready to use. Such splitting of the disk/transducer 100 advantageously facilitates ease of access to the center opening 108 for inserting or removing the sterile tube 130 and for removing the disk/transducer 100 from the needle 140 after the needle has been inserted into the patient.

In use, a user can take the disk/transducer 100 and place it at the surgically prepped desired region of the patient for the particular vascular or other access. Sterile ultrasonic coupling gel may be used between the disk/transducer 100 and the skin of the patient. The user can scan the area under the transducer 100 by manipulating the position, location and the angle of the disk/transducer 100 pressed against the skin. The image on the monitor can display the underlying structures with the cross-hair symbol 210 hovering in the center of the image if the disk/transducer 100 is properly aligned with the underlying vessel 120 or structure. Once the suitable insertion point is identified, the user can insert the needle 140 through the sterile channel 130 in the center of the disk/transducer 100 and advance it until the lumen of the vessel 120 or other desired structure is reached. The reaching can be confirmed by a sensor signal in the needle 140, or by the withdrawal of blood or fluid through the needle. Once the insertion is confirmed, the disk/transducer 100 can be opened (i.e., the split portions separated from each other), decoupled from the needle 140 and removed from the field.

Having thus described several exemplary embodiments of the disclosure, it is to be appreciated various alterations, modifications, and improvements will readily occur to those skilled in the art. For example, a computer-enhanced image of the target vessel, organ or other structure, such as the image in display 200 described above with respect to FIGS. 2B, 3B and 4B, can simplify insertion decision making process for the user and reduce the amount of additional training required to perform the procedure. Two- or three-dimensional color or black and white graphic representations of the structures in the region can, in some embodiments, replace conventional black and white grainy ultrasonic images. In some embodiments, gender and weight specific databases of the structures for the particular region (e.g., femoral triangle, neck, subclavian region) can be pre-loaded to a memory of a logic unit or processor. After the user keys in the insertion region and inputs patient gender and weight, stored data can be preloaded as a base matrix. Real-time ultrasound generated data can be then incorporated into the matrix and imaged into the graphic on the screen.

Accordingly, the foregoing description and drawings are by way of example only.

What is claimed is:

1. An ultrasound probe system for guiding introduction of an instrument into a patient, comprising:
a disk having a surface and an open channel passing axially through the disk, the open channel being configured to receive the instrument therethrough to permit alignment of the instrument with respect to the disk; and
at least two ultrasound transducer elements disposed on the surface of the disk in a parallel arrangement, each of the at least two ultrasound transducer elements being configured to transmit and receive ultrasonic waves for detecting an anatomical structure.

2. The system of claim 1, wherein an axis of the open channel is substantially perpendicular to the surface of the disk.

3. The system of claim 1, wherein an axis of the open channel is at an angle of between zero and 90 degrees with respect to the surface of the disk.

4. The system of claim 1, wherein the disk is configured to accept a sheet or tube within the open channel.

5. The system of claim 1, wherein the disk is split into at least two separable portions.

6. The system of claim 5, wherein the at least two separable portions are hingedly attached to each other.

7. The system of claim 1, further comprising a processor operatively coupled to the at least two ultrasound transducer elements and configured to:
receive one or more signals from each of the at least two ultrasound transducer elements;
detect the anatomical structure based on the one or more signals; and
generate a graphical representation of a position of the anatomical structure with respect to each of the at least two ultrasound transducer elements.

8. The system of claim 7, wherein the processor is further configured to calculate a degree of alignment of the at least two ultrasound transducer elements with respect to a longitudinal axis of the anatomical structure.

9. The system of claim 8, wherein the processor is further configured to generate a graphical cross-hair or other marker aligned with respect to the graphical representation of the position of the anatomical structure based at least in part on the calculated degree of alignment.

10. A method of guiding introduction of an instrument into a patient, the method comprising:
receiving one or more signals from each of at least two ultrasound transducer elements disposed in a parallel arrangement on a disk having a surface and an open channel passing axially through the disk, the open channel being configured to receive the instrument therethrough to permit alignment of the instrument with respect to the disk;
detecting the anatomical structure based on the one or more signals; and
generating a graphical representation of a position of the anatomical structure with respect to each of the at least two ultrasound transducer elements.

11. The method of claim 10, further comprising calculating a degree of alignment of the at least two ultrasound transducer elements with respect to a longitudinal axis of the anatomical structure.

12. The method of claim 11, further comprising generating a graphical cross-hair or other marker aligned with respect to the graphical representation of the position of the anatomical structure based at least in part on the calculated degree of alignment.

13. The method of claim 12, further comprising:
receiving a second signal from a sensor located at or near the tip of the instrument; and
generating a graphical indication of a distance between the tip of the instrument and the anatomical structure based at least in part on the second signal.
